SEQUENCE LISTING (8 sequences)

SEQ ID NO: 1 - protein, 15 aa, Artificial Sequence
  Synthetic Peptide
KVKFMDVYQRSYCHP

SEQ ID NO: 2 - protein, 15 aa, Artificial Sequence
  Synthetic Peptide
KLTFMELYQLKYKGI

SEQ ID NO: 3 - protein, 15 aa, Artificial Sequence
  Synthetic Peptide
KLTWMELYQLAYKGI

SEQ ID NO: 4 - protein, 15 aa, Artificial Sequence
  Synthetic Peptide
KLTWKELYQLAYKGI

SEQ ID NO: 5 - protein, 15 aa, Artificial Sequence
  Synthetic Peptide
KLTWMELYQLKYKGI

SEQ ID NO: 6 - protein, 15 aa, Artificial Sequence
  Synthetic Peptide
KLTWQELYQLAYKGI

SEQ ID NO: 7 - protein, 15 aa, Artificial Sequence
  Synthetic Peptide
KLTWKELYQLKYKGI

SEQ ID NO: 8 - protein, 15 aa, Artificial Sequence
  Synthetic Peptide
KLTWQELYQLKYKGI

(19) United States
(12) Patent Application Publication
(10) Pub. No.: US 2009/0305994 A1
(43) Pub. Date: Dec. 10, 2009

(54) Compounds Modulating Vegf Receptor and Uses Thereof

(76) Inventors: Lucas Domenico D'Andrea, Napoli (IT); Carlo Pedone, Napoli (IT); Bruno Trimarco, Napoli (IT); Guido Iaccarino, Napoli (IT); Maria Caterina Turco, Avellino (IT); Arturo Leone, Milano (IT); Patrizia Roselli, Napoli (IT); Marina Leone, Napoli (IT); Stefania Leone, Napoli (IT)

Correspondence Address: YOUNG & THOMPSON, 209 Madison Street, Suite 500, ALEXANDRIA, VA, 22314, US

(21) Appl. No.: 11/988,094

(22) PCT Filed: Jun. 29, 2006
(86) PCT No.: PCT/EP2006/006314

§ 371 Date: Aug. 6, 2008

Related U.S. Application Data
(60) Provisional application No. 60/694,671, filed on Jun. 29, 2005.

(51) Int. Cl. A61K 38/10; A61P 9/00; A61P 35/00

(52) U.S. Cl. 514/14

(57) ABSTRACT

The present invention is related to the use of compounds which bind to the Vascular Endothelial Growth Factor Receptors and modulate the angiogenic response. The compounds, which mimic the VEGF amino acid region 17-25 involved in receptor recognition thereby inhibiting or stimulating the angiogenic process, can be used in the treatment of diseases characterized by excessive or defective angiogenesis VEGF-dependent, such as chronic ischemia, cancer, proliferative retinopathy and rheumatoid arthritis, states or conditions benefiting from the formation or regeneration of new vessels, as well as in the diagnosis of pathologies which present a overexpression of VEGF receptors or as biochemical tools to analyze the cellular pathways dependent on VEGF receptor activation.

The present invention concerns the use of compounds which interact with the VEGF receptor and modulate the VEGF dependent biological response. The compounds are related to a specific region of VEGF, the helix region 17-25, which is involved in receptors binding. Specifically, the invention provides the use of these compounds for the treatment of pathologies related to the modulation of the VEGF biological activity, for the diagnosis of pathologies which present overexpression of VEGF receptors and as biochemical tools for the study of cellular pathways dependent on the activation of VEGF receptors.

### BACKGROUND OF THE INVENTION

Angiogenesis is a physiological process which refers to the remodeling of the vascular tissue characterized by the branching out of a new blood vessel from a pre-existing vessel. It is intimately associated with endothelial cell (EC) migration and proliferation. ECs are particularly active during embryonic development while during adult life EC turnover is very low and limited to particular physiological phenomena (Carmeliet, P. Nat Med 2003, 9, 653). In a healthy individual angiogenesis is finely tuned by pro- and anti-angiogenic factors, the shift from this equilibrium (angiogenic switch), under specific stimuli such as hypoxia, is related to several human diseases (pathological angiogenesis) (Hanahan, D., Folkman, J. Cell 1996, 86, 353). The prevalence of pro-angiogenic factors (excessive angiogenesis), is associate with cancer, proliferating retinopathy, rheumatoid arthritis and psoriasis. Whereas, insufficient angiogenesis is at the basis of coronary diseases, ischemia and a reduced capacity for tissue regeneration (Carmeliet, P., Jain, R. K. Nature 2000, 407, 249).

A number of clinical studies have shown that angiogenesis is an essential process for the growth of solid tumors. The suppression of any phases of angiogenesis inhibits the formation of new vessels thus influencing the growth of the tumor and the generation of metastases. Tumor cells, as normal tissues, need to receive oxygen and metabolites to survive. Initially, when the neoplastic lesion is small (diameter less than 2 mm), the tumor is able to receive these substances through diffusion (avascular phase) and it can remain dormant reaching a stationary state between proliferation and apoptosis. Successively (vascular phase), when tumor cells begin to duplicate indiscriminately, they induce a shift in the equilibrium between pro- and anti-angiogenic factors (angiogenic switch), promoting the formation of a vascular network in order to satisfy the growing need of oxygen and nutrients thus allowing the exponential growth of the tumor (Hanahan, D., Folkman, J. Cell 1996, 86, 353). Moreover, the new vessels are one of the ways through which the tumor can lead to the formation of metastases.

The angiogenic switch can occur at different phases of the tumor progression, depending on the type of tumor, but, in most cases, it is a prerequisite for the growth of the tumor.

The newly formed tumor vessels show characteristics which are different from the normal one. In fact, the vessels are structurally disorganized, tortuous and dilated and they express on their membrane surface peculiar markers which can be used for the selective targeting of tumor blood vessels (Bergers, G., Benjamin, L. E. Nat Rev Cancer 2003, 3, 401; Ruoslahti, E. Nat Rev Cancer 2002, 2, 83).

Cardiovascular disease. The primary physiological response to ischemia is the local growth of capillaries. The occlusion of a major artery leads to a fall in poststenotic pressure and to a redistribution of the blood to existing arterioles. The resulting stretch and shear forces lead to the expression of endothelial chemokines, adhesion molecules and growth factors (Helisch, A., Schaper, W Z Kardiol 2000, 89, 239). The vessels undergo an immense growth process with active proliferation of both endothelial and vascular smooth muscle cells. In the case of coronary artery disease or peripheral vascular disease this angiogenic response is frequently associated with arteriogenesis. Collateral vessels can develop around the site of coronary occlusion. Although the exact mechanism of arteriogenesis is not clear, there are two distinct possibilities: to remodel the pre-existing vessels enlarging the point at which they can carry the bulk of blood flow; to involve budding of new vessels from post-capillary venules on the adventitial surface of the occluded artery that gradually expand and connect to the distal arterial segment. The excess vessels undergo apoptosis once sufficient flow has been established (Simons, M., Ware, J. A. Nat Rev Drug Discov 2003, 2, 863).

It is very important for individuals to have the ability to form a good collateral circulation and to increase capillary bed size in order to compensate after an ischemic insult and thus limiting the damage (Schaper, W., Ito, W. D. Circ Res 1996, 79, 911; Helisch, A., Schaper, W. Microcirculation 2003, 10, 83). It is not uncommon that individuals with peripheral artery disease, in spite of extensive lower extremity arterial occlusions, remain nearly asymptomatic because of a naturally robust collateral network (Helisch, A., Schaper, W. Z Kardiol 2000, 89, 239). Because the degree of collateral blood vessels formation in chronic ischemia is different from an individual to another it is important to elucidate the basis of the interindividual differences in the angiogenic response (Schultz, A. et al. Circulation 1999, 100, 547). Several observations suggest that the genetic background may at least in part account for the lack of collateral development during chronic coronary artery disease.

Experimental data shows that hypoxic induction of VEGF is significantly reduced in patients with poor collateral development (Schultz, A. et al. Circulation 1999, 100, 547). Individual variations in the potential for endogenous neovascularization are not likely limited to upstream deregulation of hypoxia inducing factor-1 (HIF-1) mediating VEGF expression. Defective expression of tissue metalloproteinases, tissue plasminogen activators, or other components of the cascade responsible for neovascularization, including variations in intracellular signaling may prove to be contributory (Isner, J. M. J Clin Invest 2000, 106, 615).

Angiogenesis is mainly regulated by the Vascular Endothelial Growth Factor (VEGF). VEGF is a mitogen specific for endothelial cells and in the last years many efforts have been pursued to modulate the angiogenic response targeting VEGF and its receptors.

Vascular Endothelial Growth Factor (VEGF) is a potent angiogenic factor, a mitogen specific for vascular endothelial cells and plays a major role in angiogenesis. VEGF and its receptors are overexpressed in pathological angiogenesis making this system a potential target for therapeutic and diagnostic applications (Hanahan, D., Folkman, J. Cell 1996, 86, 353; Carmeliet, P., Jain, R. K. Nature 2000, 407: 249).

VEGF is a homodimeric protein belonging to the cystine knot growth factor family. It is encoded by a single gene which is expressed in four different isoforms (VEGF₁₂₁, VEGF₁₆₅, VEGF₁₈₉, VEGF₂₀₅) due to different splicing events. VEGF₁₆₅, the most abundant isoform, is a 45 KDa glycoprotein and it binds to heparin with high affinity. The biological function of VEGF is mediated through binding to two tyrosine kinase receptors, the kinase domain receptor (KDR, Flk-1 or VEGFR-2) and the Fms-like tyrosine kinase (Flt-1 or VEGFR-1). VEGF induces receptor dimerization which stimulates endothelial cell mitogenesis. KDR and Flt-1 are localized on the cell surface of various endothelial cell types (Ferrara, N. et al., Nat Med 2003, 9, 669). Increased expression of these receptors occurs in response to several stimuli and results in priming of endothelial cells towards cell proliferation, migration and angiogenesis (Brogi, E. et al., J Clin Invest. 1996, 97, 469).

Different mechanisms have been shown to be involved in the regulation of VEGF gene expression. Among these, oxygen tension plays a major role. VEGF mRNA expression is rapidly and reversibly induced by exposure to low oxygen pressure in a variety of normal and transformed cultured cell types (Abedi, H. & Zachary, I. J Biol Chem 1997, 272, 15442).

The role of VEGF in different pathologies has been reported and blocking the interaction of VEGF with its receptors has been demonstrated to have several therapeutic applications. Many reviews and patents describe the role ed the usage of VEGF in pathological angiogenesis and discuss its therapeutic applications. All patent applications, patents and publications cited are hereby incorporated by reference in their entirety.

A diseases which can benefit form a therapy based on the inhibition of the interaction between VEGF and its receptors is cancer (D. J. Hicklin & L. M. Ellis J. Clin. One. 2005, 23, 1011; N. Ferrara et al., Nat. Med. 2003, 9, 669; N. Ferrara & T. Davis-Smyth Endocr. Rev. 1997, 18, 4). VEGF is overexpressed in several type of tumors (lung, thyroid, breast, gastrointestinal, kidney, ovary, uterine cervix, carcinomas, angiosarcomas, germ cell tumors, intracranial). VEGF receptors are overexpressed in some type of tumors, such as, non-small-cell lung carcinoma, melanoma, prostate carcinoma, leukemia, mesothelioma, breast carcinoma (D. J. Hicklin & L. M Ellis J. Clin. One. 2005, 23, 1011), and on the surface on angiogenically active endothelial cells.

VEGF is implicated in intraocular neovascularization which may lead to vitreous hemorrhage, retinal detachment, neovascular glaucoma (N. Ferrara et al., Nat. Med. 2003, 9, 669; N. Ferrara Curr. Opin. Biotech. 2000, 11, 617) and in eye disorders such as age related macular degeneration and diabetic retinopathy (US 2006/0030529).

VEGF is also implicated in the pathology of female reproductive tract, such as ovarian hyperstimulation syndrome and endometriosis.

VEGF has been implicated in psoriasis, rheumatoid arthritis (P. C. Taylor Arthritis Res 2002, 4, S99) and in the development of brain edema.

Diseases caused by a defective angiogenesis can be treated (therapeutic angiogenesis) with agents able to promote the growth of new collateral vessels. The VEGF-induced angiogenesis has several therapeutic applications. Of course, molecules which bind to VEGF receptors and mimic the biological activity of VEGF are useful for the treatment of these diseases.

VEGF has been used for the treatments of ischemic cardiovascular diseases to stimulate the revascularization in ischemic regions, to increase coronary blood flow and to prevent restenosis after angioplasty. (M Simons & J. A. Ware Nat. Rev. Drug Disc. 2003, 2, 1; N. Ferrara & T. Davis-Smyth Endocr. Rev. 1997, 18, 4).

VEGF and its receptors have been implicated in stroke, spinal cord ischemia, ischemic and diabetic neuropathy. VEGF is a therapeutic agent for the treatment of neuron disorders such as Alzheimer disease, Parkinson's disease, Huntington disease, chronic ischemic brain disease, amyotrophic later sclerosis, amyotrophic later sclerosis-like disease and other degenerative neuron, in particular motor neuron, disorders (US 2003/0105018; E. Storkebaum & P. Carmeliet J. Clin. Invest. 2004, 113, 14).

VEGF has a basic role in bone angiogenesis and endochondral bone formation. These findings suggest that VEGF may be useful to promote bone formation enhancing revascularization. Conditions which can benefit from a treatment with VEGF are bone repair in a fractures, vertebral body or disc injury/destruction, spinal fusion, injured meniscus, avascularnecrosis, cranio-facial repair/reconstruction, cartilage destruction/damage, osteoarthritis, osteosclerosis, osteoporosis, implant fixation, inheritable or acquired bone disorders or diseases (US2004/0033949).

VEGF has been implicated in the process of gastric ulcer (Ma et al., Proc. Natl. Acd. Sci. USA 2001, 98, 6470) wound healing, diabetic foot ulcers and diabetic neuropathy.

VEGF has been implicated in neurogenesis (K Jin et al., Proc. Natl. Acd. Sci. USA 2002, 99, 11946) and for the treatment of pathological and natural states benefiting from the formation or regeneration of new vessels (US 2005/0075288).

VEGF or molecules able to bind to VEGF receptors can be useful for the diagnosis of pathologies which present a overexpression of VEGF receptors (Li et al., Annals of Oncology 2003, 14, 1274) and to imaging angiogenic vasculature (Miller et al., J. Natl. Cancer Inst. 2005, 97, 172).

Molecular agents for imaging angiogenesis must bind to the VEGF receptors with high specificity and be detectable at low concentrations. They should be labeled according to the imaging modalities, PET, SPECT, and, to a lesser extent, ultrasound (with microbubble contrast agents) and optical imaging (with fluorescent contrast agents). In addition, even though the sensitivity of MRI is low, molecular imaging of angiogenesis is possible with oligomerized paramagnetic substances linked to an agent, that binds a molecular marker of angiogenesis (Miller et al., J. Natl. Cancer Inst. 2005, 97, 172).

Several VEGF structures have been reported so far: VEGF free (Muller, Y. A et al.,) Structure 1997, 5, 1325; Muller, Y. A. et al., Proc Natl Acad Sci USA 1997, 94, 7192), in complex with an antibody (Muller, Y. A. et al., Structure 1998, 6, 1153), with peptide inhibitors (Wiesmann, C. et al., Biochemistry 1998, 37, 17765; Pan, B. et al., J Mol Biol 2002, 316, 769) and with the Flt-1 domain 2 (Wiesmann, C. et al., Cell 1997, 91, 695). Two VEGF monomers, linked by disulfide bonds, bind to two receptor molecules which are localized at the poles of the VEGF antiparallel homodimer. The overall structure of the complex possesses approximately a two-fold symmetry. The analysis of structural and mutagenesis data allowed to identify the residues involved in the binding to the receptors. KDR and Flt-1 share the VEGF binding region, in fact 5 out of 7 most important binding residues are present in both interfaces. The segments of VEGF₈₋₁₀₉ in contact with Flt-1_{D2} include residues from the N-terminal helix (17-25), the loop connecting strand β3 to β4 (61-66) and strand β7 (103-106) of one monomer, as well as residues from strand β2 (46-48) and from strands β5 and β6 together with the connecting turn (79-91) of the other monomer. The recognition interface is manly hydrophobic, except for the polar interaction between Arg224 (Flt-1) and Asp63 (VEGF) (Wiesmann, C. et al., Cell 1997, 91, 695).

Many approaches have been pursued to modulate the VEGF-receptors interaction and new molecular entities as peptides (Keyt, B. A. et al., J Biol Chem 1996, 271, 5638; An, P. et al., Int J Cancer 2004, 111, 165; Scheidegger, P. et al., Biochem J 2001, 353, 569; Jia, H. et al., Biochem Biophys Res Commun 2001, 283, 164; Binetruy-Tournaire, R. et al., Embo J 2000, 19, 1525. Hetian, L. et al., J Biol Chem 2002, 277, 43137; Zilberberg, L. et al., J Biol Chem 2003, 278, 35564; El-Mousawi, M, et al., J Biol Chem 2003, 278, 46681-46691.) and antibodies (Prewett, M et al., Cancer Res 1999, 59, 5209; Cooke, S. P. et al., Cancer Res 2001, 61, 3653) have been reported to bind to the extracellular region of the VEGF receptors. A large number of them showed an antagonist activity and only few behave as agonists (An, P. et al., Int J Cancer 2004, 111, 165).

### DESCRIPTION OF THE INVENTION

The invention relates to compounds mimetic of the VEGF helix region spanning VEGF sequence from Phe17 to Tyr25 (hereafter “VEGF-helix 17-25 mimetic compound”), said compounds being able to recognize VEGF receptors and to modulate both endothelial cell proliferation and angiogenesis or propensity towards angiogenesis, and to their use in the preparation of an agent or composition for the treatment of states, diseases or conditions that benefit from the formation or regeneration of vessels.

In a preferred embodiment said compounds are peptides selected for the group consisting of SEQ ID No.1 through SEQ ID No.8, according to the following Table 1

TABLE 1

| | |
|---|---|
| (SEQ ID No. 1) | KVKFMDVYQRSYCHP |
| (SEQ ID No. 2) | KLTFMELYQLKYKGI |
| (SEQ ID No. 3) | KLTWMELYQLAYKGI |
| (SEQ ID No. 4) | KLTWKELYQLAYKGI |
| (SEQ ID No. 5) | KLTWMELYQLKYKGI |
| (SEQ ID No. 6) | KLTWQELYQLAYKGI |
| (SEQ ID No. 7) | KLTWKELYQLKYKGI |
| (SEQ ID No. 8) | KLTWQELYQLKYKGI |

A preliminary characterization in water by Nuclear Magnetic Resonance and circular dichroism showed a good propensity for a helix conformation for these peptides. No biological activity was reported for these compounds (L. D. D'Andrea et al., Peptides 2002 Edizioni Ziino, Napoli, Italy (2002), 454).

The inventors have characterized in vitro and in vivo the biological behavior of these peptides. Some of them bind to the VEGF receptors and show a VEGF-like biological activity, others bind to the VEGF receptors and act as VEGF antagonist. Based on their biological properties, these compounds can be used for the treatment of pathologies related to the modulation of the VEGF biological activity, for the diagnosis of pathologies which present a overexpression of VEGF receptors and as biochemical tools for the study of cellular pathways dependent on the activation of VEGF receptors.

Preferably the compounds are used for the diagnosis and treatment of pathologies relating to angiogenesis, such as chronic ischemia, cancer, proliferative retinopathy and rheumatoid arthritis. In particular, compounds which stimulate the angiogenesis are used for the treatment of states, conditions or diseases that may benefit from the formation or regeneration of blood vessels.

According to one embodiment the present invention provides the use of a VEGF helix 17-25 mimetic compound, which is preferably a peptide selected from SEQ ID No.1 to SEQ ID No.8, as a therapeutic agent for the treatment of cancer, preferably of tumors which express on their surface VEGF receptors and tumors dependent on angiogenesis such as lung tumors, thyroid tumor, breast cancer, gastrointestinal tumors, kidney tumors, ovary tumors, uterine cervix tumor, carcinomas, angiosarcomas, germ cell tumors, intracranial tumors.

In another embodiment the invention provides the use of a VEGF-helix 17-25 mimetic compound, which is preferably a peptide selected from SEQ ID No. 1 to SEQ ID No.8, as a therapeutic agent for the treatment of eye disorders such as age related macular degeneration, diabetic retinopathy, vitreous hemorrhage, retinal detachment, neovascular glaucoma.

In another embodiment the invention provides the use of a VEGF-helix 17-25 mimetic compound, which is preferably a peptide selected from SEQ ID No.1 to SEQ ID No.8, as a therapeutic agent for the treatment of pathologies of female reproductive tract, such as ovarian hyperstimulation syndrome and endometriosis.

In another embodiment the invention provides the use of a VEGF-helix 17-25 mimetic compound, which is preferably a peptide selected from SEQ ID No.1 to SEQ ID No.8, as a therapeutic agent for the treatment of psoriasis.

In another embodiment the invention provides the use of a VEGF-helix 17-25 mimetic compound, which is preferably a peptide selected from SEQ ID No.1 to SEQ ID No.8, as a therapeutic agent for the treatment of rheumatoid arthritis.

In another embodiment the invention provides the use of a VEGF-helix 17-25 mimetic compound, which is preferably a peptide selected from SEQ ID No.1 to SEQ ID No.8, as a therapeutic agent for the treatment of brain edema.

In another embodiment the invention provides the use of a VEGF-helix 17-25 mimetic compound, which is preferably a peptide selected from SEQ ID No.1 to SEQ ID No.8, as a therapeutic agent for the treatment of ischemic cardiovascular diseases.

In another embodiment the invention provides the use of a VEGF-helix 17-25 mimetic compound, which is preferably a peptide selected from SEQ ID No.1 to SEQ ID No.8, as a therapeutic agent for the treatment of neuronal disorders, particularly Alzheimer disease, Parkinson's disease, Huntington disease, chronic ischemic brain disease, amyotrophic lateral sclerosis, amyotrophic lateral sclerosis-like disease and other degenerative neuronal, in particular motor-neuron disorders.

In another embodiment the invention provides the use of a VEGF-helix 17-25 mimetic compound, which is preferably a peptide selected from SEQ ID No.1 to SEQ ID No.8, as a therapeutic agent to induce bone formation and to treat bone defects, preferably vertebral body or disc injury/destruction, spinal fusion, injured meniscus, avascularnecrosis, cranio-facial repair/reconstruction, cartilage destruction/damage, osteoarthritis, osteosclerosis, osteoporosis, implant fixation, inheritable or acquired bone disorders or diseases.

In another embodiment the invention provides the use of a VEGF-helix 17-25 mimetic compound, which is preferably a peptide selected from SEQ ID No.1 to SEQ ID No.8, as a therapeutic agent for the treatment of gastric ulcer.

In another embodiment the invention provides the use of a VEGF-helix 17-25 mimetic compound, which is preferably a peptide selected from SEQ ID No.1 to SEQ ID No.8, as a therapeutic agent for the treatment of diabetic foot ulcers.

In another embodiment the invention provides the use of a VEGF-helix 17-25 mimetic compound, which is preferably a peptide selected from SEQ ID No.1 to SEQ ID No.8, as therapeutic agent for diabetic neuropathy.

In another embodiment the invention provides the use of a VEGF-helix 17-25 mimetic compound, which is preferably a peptide selected from SEQ ID No.1 to SEQ ID No.8, as an agent to stimulate neuroangiogenesis.

In another embodiment the invention provides the use of a VEGF-helix 17-25 mimetic compound, which is preferably a peptide selected from SEQ ID No.1 to SEQ ID No.8, as a therapeutic agent for wound healing.

In another embodiment the invention provides the use of a VEGF-helix 17-25 mimetic compound, which is preferably a peptide selected from SEQ ID No.1 to SEQ ID No.8, as a therapeutic agent for treatment of pathological and natural states benefiting from the formation or regeneration of blood vessels.

In another embodiment the invention provides the use of a VEGF-helix 17-25 mimetic compound, which is preferably a peptide selected from SEQ ID No.1 to SEQ ID No.8, for the diagnosis of pathologies which present a overexpression of VEGF receptors.

In another embodiment the invention provides the use of a VEGF-helix 17-25 mimetic compound, which is preferably a peptide selected from SEQ ID No.1 to SEQ ID No.8, for the imaging of angiogenic vasculature.

In another embodiment the invention provides the use of a VEGF-helix 17-25 mimetic compound, which is preferably a peptide selected from SEQ ID No. 1 to SEQ ID No.8, as a biochemical tool for the study of cellular pathways dependent on the activation of VEGF receptors.

### BRIEF DESCRIPTION OF THE DRAWINGS

FIG. 1

VEGF receptors binding and activation. a) VEGF competitive binding on BAEC. 1 μg of membrane protein was plated with QK and [¹²⁵I]-VEGF (500000 cpm, 10⁻¹⁰ M). b) KDR activation. After stimulation total KDR was immunoprecipitated from a whole-cell protein extracts and phospho-tyrosine was visualized by a specific antibody, anti-rabbit HRP-conjugated secondary antibody and standard chemiluminescence. c) Flt-1 activation. After stimulation total Flt-1 was immunoprecipitated from a whole-cell protein extracts and phospho-tyrosine was visualized by specific antibody, anti-rabbit HRP-conjugated secondary antibody and standard chemiluminescence.

FIG. 2

Effect of QK and VEGF15 on ERK1/2 activation. Serum deprived BAEC were treated with QK (a) or with VEGF 15 (b) in absence or in presence of VEGF₁₆₅ (100 ng/ml) for 15 minutes at 37° C. and then dissolved in RIPA-SDS buffer. Total ERK1/2 and the phosphorylated form of ERK1/2 were visualized by specific antibodies.

FIG. 3

Effect of QK on cell proliferation. a) DNA synthesis. BAEC were incubated in DMEM with [³H]-thymidine and QK in absence or in presence of VEGF₁₆₅ (100 ng/ml). After 24 hours cells were fixed and lysed. Scintillation liquid was added and [³H]-thymidine incorporation was evaluated. b) Cell proliferation. BAEC were stimulated with the indicated amount of QK in absence or in presence of VEGF₁₆₅ (100 ng/ml). Cell number was determined at 24 hours after stimulation. c) RB phosphorylation. p-RB was evaluated at 12 and 18 hours after stimulation with QK (10-6M), VEGF₁₆₅ (100 ng/ml) and VEGF 15 (10⁻⁶ M).

FIG. 4

In vitro angiogenic properties of QK. Human endothelial cells were co-cultured with other human cells in a specially designed medium in a 24 well plate. Every three days, QK alone or a combination of QK and VEGF₁₆₅ (100 ng/ml) was added to the cultures. On the eleventh day, cells were fixed with ice cold 70% ethanol and tubule formation was visualized by staining for anti-human CD31 (PECAM-1). Sample images are reported in the inserts a-d. a) Suramine (20 μM) and b) VEGF₁₆₅ were used as negative and positive control respectively. e) The number of cellular connections and the total tubule length were determined using a software which analyze the images after digitalization.

FIG. 5

Blood Flow evaluation in vivo. The increased neoangiogenetic responses by QK and VEGF intraarterial chronic infusion during chronic ischemia in vivo was evaluated. (a) TIMI Frames count (FC). After 15 days of chronic ischemia digital angiographies evidenced a reduced number of TIMI FCs in ischemic hind-limbs treated with QK and VEGF respect to sham treated rats used as controls (*: p<0.05). (b) Dyed beads dilution. Similarly, QK and VEGF ameliorates blood flow in ischemic hindlimb respect to controls (*: p<0.05).

FIG. 6

HUVE cells (1×104/cm2) were incubated in medium without FBS, in the absence or presence of VEGF (20 ng/ml) and QK (5 ng/ml), at 37° C. in a 5% CO₂ atmosphere. After 4 h, caspase 3 activity was determined. Results are expressed as mean values of triplicates.

FIG. 7

HUVE cells (1×104/cm2) were incubated in medium without FBS, in the absence or presence of VEGF (20 ng/ml) and the indicated peptides (20 ng/ml), at 37° C. in a 5% CO₂ atmosphere. After 4 h, caspase 3 activity was determined. Results are expressed as mean values of triplicates.

FIG. 8

HUVE cells (1×104/cm2) were incubated with 500 nM MA peptide conjugated with fluorescein and competed with increasing amount of VEGF at 30 min at 4° C. in the dark. Then, cell fluorescence was analyzed by flow cytometry.

FIG. 9

(a) HUVE cells (1×104/cm2) were incubated whit VEGF (20 ng/ml) in the absence or presence of MA peptide (100 ng/ml), in duplicates, for 30 min. Then cell lysates were obtained and analysed in Western blot with anti-phospho-ERK antibody. b) HUVE cells (1×104/cm2) were incubated with VEGF (20 ng/ml) in the absence or presence of MA peptide (100 ng/ml), in triplicates, for 24 h, at 37° C. in a 5% CO₂ atmosphere. Then FITC-Annexin V binding was analyzed by flow cytometry.

### EXAMPLES

### Example 1

Biological assays in vitro and on bovine aorta endothelial cells (BAEC) suggested that the peptide in table 1 with the SEQ ID No.8 (namely “QK”) is able to bind to the VEGF receptors and to compete with iodinated VEGF₁₆₅ possibly targeting the same region on the receptor. The natural peptide SEQ ID No.1 (VEGF15) does not bind to the receptor meaning that the helical structure is necessary for the biological activity. Furthermore, QK induced endothelial cells proliferation, activated signaling induced by VEGF₁₆₅ and increased the VEGF biological response. QK was able to induce capillary formation and organization in an in vitro assay on matrigel substrate and angiogenesis in vivo.

These results provide evidence for the 17-25 helix region of VEGF to be involved in VEGF receptor activation. Peptides designed to resemble this region share numerous biological properties of VEGF₁₆₅, thus suggesting that this region is of potential interest for biomedical applications and molecule mimicking this region could be attractive for therapeutic and diagnostic applications (L. D. D'Andrea et al., Proc. Natl. Acad. Sci. USA (2005), 102, 14215).

Peptide Synthesis. Peptides were synthesized on solid phase using Rink Amide MBHA resin (Novabiochem) with standard Fmoc (N-(9-Fluorenyl)methoxycarbonyl) chemistry. The N-terminal lysine was protected with the methyltrytil group to allow selective deprotection and peptide labeling. Cleavage from the resin were achieved by treatment with trifluoracetic acid, triisopropyl silane, water, (95; 2.5; 2.5) at room temperature for 3 hours. Purity and identity of the peptides were assessed by HPLC and MALDI-ToF mass spectrometry.

Cell culture. EC from bovine aorta, immortalized with SV40, were cultured in DMEM (Sigma) supplemented with 10% FBS (Invitrogen) at 37° C. in 95% air-5% CO₂. In all the experiments VEGF₁₆₅ (Alexis) was used at 100 ng/ml.

VEGF receptors binding assay. Cells were homogenized in lysis buffer (12.5 mM Tris pH 6.8, 5 mM EDTA, 5 mM EGTA) and membranes were separated from the cytosol fraction by centrifugation. Membranes were suspended in binding buffer (75 mM Tris, 12.5 mM MgCl₂, 2 mM EDTA) and an equal amount of membrane protein (1 μg) was plated in 96 well plates with QK (10⁻¹³ to 10⁻⁸ M) and [¹²⁵I]-VEGF (Amersham). VEGF binding was evaluated with a y-counter.

Western blot. Cells were plated on six-well dishes and serum starved overnight. On the next day, cells were treated with different amount of peptide in absence or in presence of VEGF₁₆₅ for 15 minutes at 37° C. and then dissolved in RIPA-SDS buffer (50 mM Tris-HCl (pH 7.5), 150 mM NaCl, 1% NP-40, 0.25% deoxycholate, 9.4 mg/50 ml sodium orthovanadate, 20% sodium dodecyl sulphate). In some experiments, total KDR and Flt-1 were immunoprecipitated from an equal amount of whole-cell protein extracts using protein A/G agarose beads conjugated with antibodies raised against total KDR or Flt-1 (R&D). Proteins from whole-cell extracts or immunocomplexes were resolved by PAGE and transferred to nitrocellulose. Total extracellular signal-regulated kinase 1 and 2 (ERK1/2), serine-tyrosin phosphorylated ERK1/2, phospho-tyrosine (Cell signaling) and phospho-RB (p-RB) (Santacruz) were visualized by specific antibodies, anti-rabbit HRP-conjugated secondary antibody (Santacruz) and standard chemiluminescence (Pierce).

[³H]-thymidine incorporation. Cells were serum starved for 24 hours and then incubated in DMEM with [³H]-thymidine (Amersham) and QK alone (10⁻¹²-10⁻⁶ M) or with a combination of QK and VEGF₁₆₅. After 24 hours cells were fixed with trichloracetic acid (0.05%) and dissolved in NaOH 1M. Scintillation liquid was added and thymidine incorporation was evaluated with a beta counter.

Cells proliferation assay. Cells were seeded at a density of 10000 per well in six well plates, serum starved overnight and then stimulated with QK (10-12 to 10⁻⁶ M) in absence or in presence of VEGF₁₆₅. Cell number was determined at 24 hours after stimulation. The p-RB, was evaluated by western blot 12 and 18 hours after stimulation with QK (10⁻⁶ M), VEGF₁₆₅ and VEGF 15 (10⁻⁶ M).

Angiogenesis in vitro assay. Human endothelial cells were co-cultured with other human cells in a specially designed medium (Angiokit, TCS CellWorks), in a 24 well plates. Every three days, QK in absence or in presence of VEGF₁₆₅ was added to the cultures. VEGF and suramine (20 μM) were used as positive and negative controls respectively. Cells subsequently begin to proliferate and then enter a migratory phase during which they move through the matrix to form thread-like tubule structures. On the eleventh day, cells were fixed with ice cold 70% ethanol and tubule formation was visualized by staining for anti-human CD31 (PECAM-1). Results were scored with the image analysis software, Angiosys software (TCS CellWorks).

Angiogenesis In Vivo Assay

Animals and Surgical procedures. Animal studies were performed in accordance to Federico II University guidelines. Adenoviral mediated gene transfer through intravascular delivery was performed as previously described 15. In Twelve-week-old normotensive WKY, anesthetized with tiletamine (50 mg/kg) and zolazepam (50 mg/kg), we performed the ischemic hindlimb model (Ischemic neoangiogenesis enhanced by beta2-adrenergic receptor overexpression: a novel role for the endothelial adrenergic system Iaccarino et al Cir Res 2005), associated with a chronic intrafemoral artery infusion of QK (10-7 M), VEGF (10-7 M) and VEGF 15 (10-7 M) by miniosmotic pump (Cardiac βARK1 Upregulation Induced by Chronic Salt Deprivation in Rats, Iaccarino et al Hypertension 2001) (model 2002; Alzet), filled with solutions containing the substances cited and placed in the peritoneum.

Digital Angiographies and blood flow determination. After 14 days animals were anaesthetized, the catheter removed from right femoral artery and the wound closed in layers. Then the left common carotid exposed as previously described and a flame stretched PE 50 catheter was advanced into the abdominal aorta right before the iliac bifurcation, under fluoroscopic visualization (Advantix LCX, General Electrics). Maximal vasodilation was obtained by nitroglycerin (20 μg i.a.). An electronic regulated injector (ACIST Medical Systems INC) was used to deliver with constant pressure (900 psi) 0.2 ml of contrast medium (Iomeron 400, Bracco). The cineframe number for TIMI frame count assessment was measured with a digital frame counter on the suitable cine-viewer monitor as previously described. All angiograms were filmed at 5 frame/sec and were analyzed by two blinded investigators (PP, GG). TIMI frame count was done from the first frame in which the contrast medium entered iliac artery until the frame of full visualization of first paw artery bifurcation. After angiography, we injected in 108 Orange dyed beads diluted in 1 ml NaCl 0.9% (Triton Technologies) and then animals were sacrificed with a lethal dose of pentobarbital. Gastrocnemious samples of the ischemic and non ischemic HL were collected and frozen with liquid nitrogen and stored at −80° C. Next, samples were homogenized and digested, the beads were collected and suspended in DMTF. The release of dye was assessed by light absorption at 450 nm. Data are expressed as ischemic to non ischemic muscle ratio.

Histology. Tissue specimen were dissected and immediately fixed by immersion in PBS (phosphate buffered saline, 0.01M, pH 7.2-7.4)/formalin for at least 24 hours. They were then dehydrated through crescent alcohol concentration and embedded in paraffin. Five μm-thick sections were processed for histochemistry: after re-hydration, they were incubated with Bandeiraea simplicifolia I (BS-I) biotinylated lectin (Sigma, 1:50) overnight. BS-1 specific adhesion to capillary endothelium was revealed by a secondary incubation for 1 hour at room temperature with horseradish peroxidase conjugated streptavidin (Dako, 1:400), which in presence of hydrogen peroxide and diaminobenzidine gives a brown reaction product. Morphometric analysis was performed by a Leitz Diaplan microscope provided with a Leica DC 200 digital camera. Images of interest were processed by Image Pro Plus software (Media Cybernetics, MD, USA) in order to count the number of capillary blood vessels per examined area. Five to fifteen μm-thick capillary diameters were considered in this study. Five tissue sections/each animal/each experimental group were examined. The number of capillaries per 20 fields was measured on each section by two independent operators, blind to treatment (VC; GA). Mean values of the measurements from five sections/animal/experimental group were then calculated and plotted. The final values were expressed as mean capillary number/unit area equivalent to 1000 μm². The differences between groups were evaluated by Anova. For β2AR immunohistochemistry after gene transfer, muscle 6 μm-thick cryostat sections were cut and mounted on poly-L-lysine-coated slides. Sections were either kept frozen until use or fixed in cool acetone and dried. Non-specific protein-binding sites on the tissue section were blocked by incubation with normal goat serum. This was followed, without further washing, by incubation with 1:25 rabbit anti-β2AR (Santa Cruz Biotechnology, CA, USA) overnight at 4° C. An enzyme-labelled immunoreaction was carried out with a biotinylated secondary antibody followed by an avidin-conjugated alkaline phosphatase complex (Dako). Alkaline phosphatase was developed to give a red reaction product with naphthol AS-MX phosphate and new fuchsin in 0.1 M Tris/HCl buffer, pH 8.2. Immunostaining controls consisted of substituting non-immune serum for the primary antibody.

Implanted Matrigel Model in rats. Each animals was subcutaneously injected with 1 mL Matrigel Matrix High (18-22 mg/mL; Becton Dickinson, Franklin Lakes, N.J.) containing QK, VEGF 15, VEGF 165 (10-6 M) or saline solution on the back. One week later, Matrigel plugs were removed and fixed in 4% buffered formaldehyde in PBS for histologic analysis using Masson trichrome staining. The capillary-occupied area per field of view from 15 to 20 fields in tissue sections was measured using a computerized digital camera system (Olympus, Melville, N.Y.) and NIH Image 1.61 (NIH, Bethesda, Md.). The vessels are defined as those structures possessing a patent lumen and positive endothelial nuclei.

Analysis of caspase 3 activity—Cells (2×104) were lysed in a buffer containing Hepes 50 mM, DTT 1 mM, EDTA 0.1 mM, NP-40 0.1%, CHAPS 0.1% and protein quantitation determined. Protein aliquots (20 μg) were incubated with 20 μM Ac-DEVD-AMC (Pharmingen, San Diego, Calif.) in a buffer containing Hepes 50 mM, DTT 1 mM, EDTA 0.1 mM, NP-40 0.1%, CHAPS 0.1%, at 37° C. for 3 h. Caspase 3 activity was determined in the cytosolic extracts by analysing the release of 7-amino-4-methylcoumarin (AMC) from N-acetyl-DEVD-AMC (Thornberry N A, et al. Nature 1992; 356:768-74); the release of AMC was monitored in a spectrofluorometer with an excitation wavelength of 380 nm and emission wavelength of 440 nm.

Results

Peptide design: Based on the X-ray structure of the VEGF/Flt-1_{D2} complex (1FLT) (1), we designed and synthesized a peptide reproducing the VEGF binding region spanning the amino acid sequence Phe17-Tyr25. This region contains 5 (Phe17, Met18, Tyr21, Gln22, Tyr25) out of 21 residues situated at less than 4.5 Å from the receptor and it assumes, in the natural protein, an α-helix conformation. The design strategy we adopted was to keep fixed the three dimensional arrangement of the residues interacting with the receptor and stabilize the secondary structural motif. Mutagenesis data indicate that when Phe17 is mutated to Ala, the affinity towards KDR is reduced by 90-fold whereas mutations of the other four residues only slightly affect the binding (2, 3). All the five interacting residues occupy a face of the helix and they make hydrophobic interaction with the receptor. Residues on the opposite face protrude towards the protein interior and in an isolate peptide they would be solvent exposed. The helix conformation of the QK peptide was stabilized introducing N- and C-capping sequences (4), amino acid with intrinsic helix propensity and favorable electrostatic interactions (5). The capping residues were chosen based on statistical preference for each position: N′ (Leu), N_{cap}(Thr), N4 (Leu), C3 (Leu), C_{cap} (Lys), C′ (Gly) and C″ (Ile). Phe17 was replaced by Trp in order to introduce a spectroscopic probe and to increase the hydrophobic surface; Met18, which is close to the residue Asn219 of Flt-1, was substituted with a Gln residue, present in the VEGF homolog protein, Placenta Growth Factor, more suited to form favorable hydrogen bond interaction. Asp19 was replaced by Glu because of its higher helix propensity and Ser24 was substituted with Lys in order to increase helix propensity and solubility. An extra Lys residue was appended at the N-terminal to allow selective labeling. The peptide was acetylated and amidate to avoid electrostatic repulsion between peptide terminal charges and helix dipoles. The design resulted in the following QK sequence:
Ac-K₁L₂T₃Q₄K₅E₆L₇Y₈Q₉L₁₀K₁₁Y₁₂K₁₃G₁₄I₁₅—CONH₂.

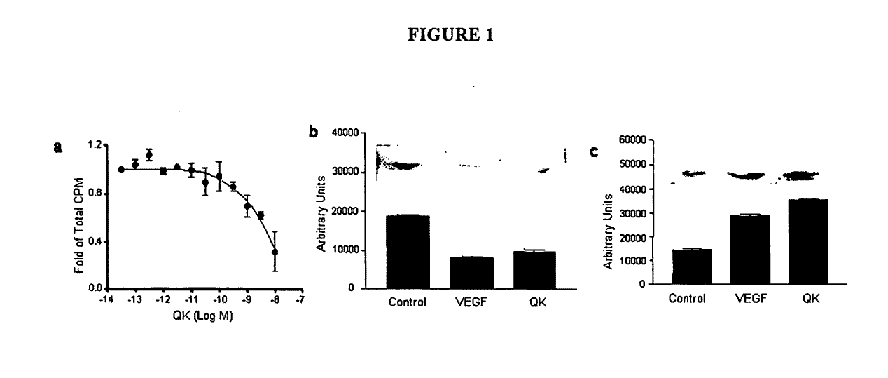

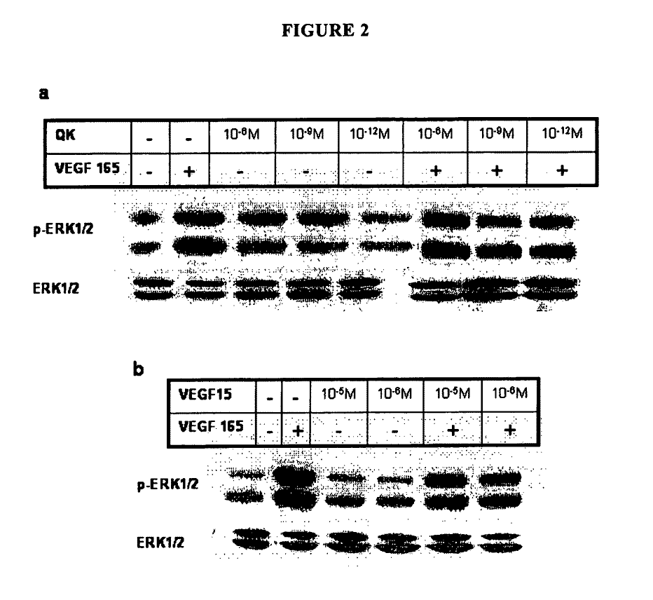

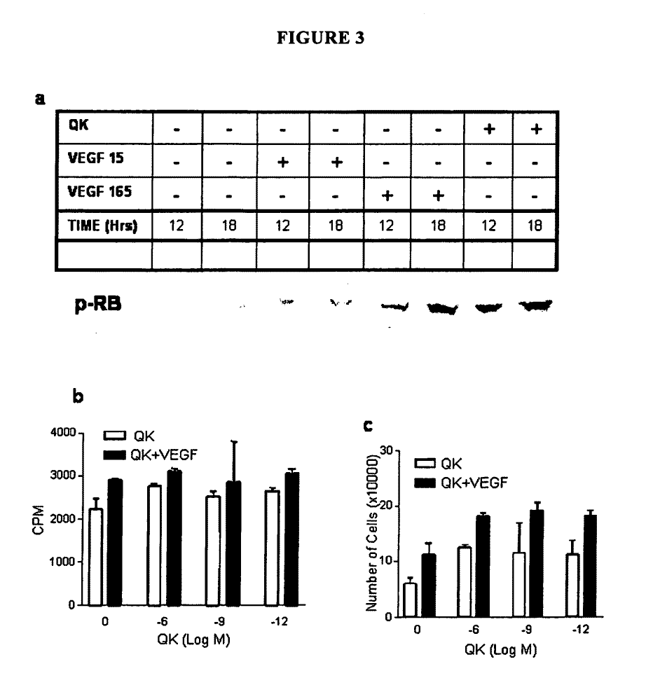

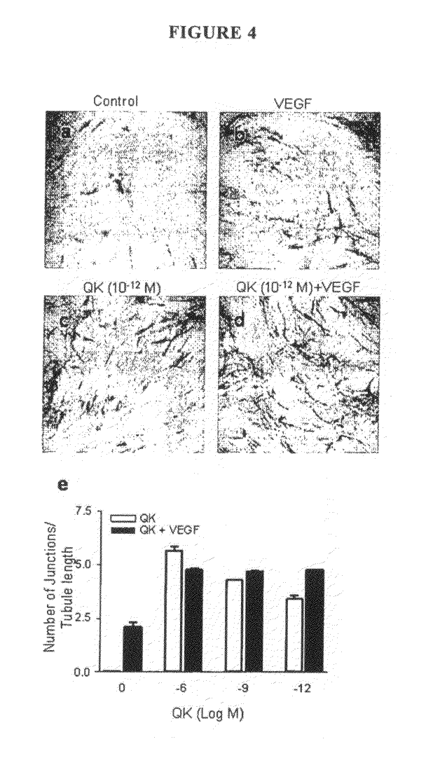

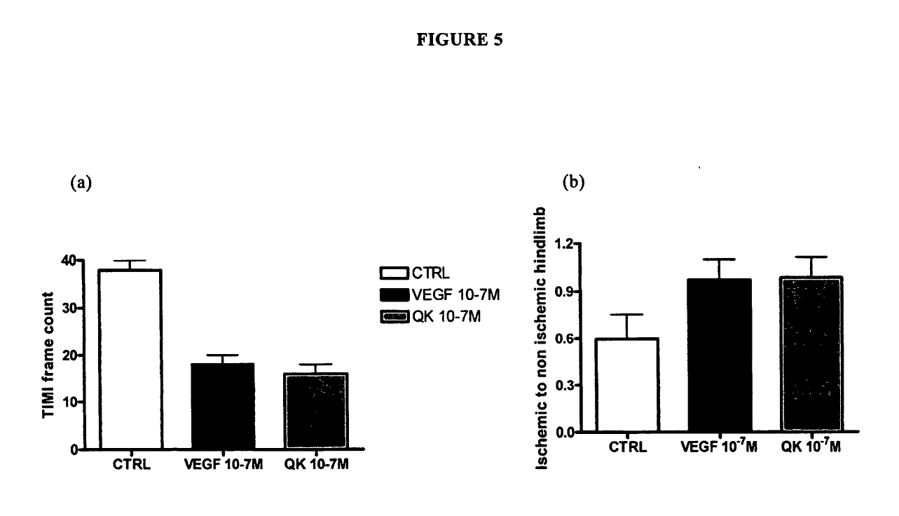

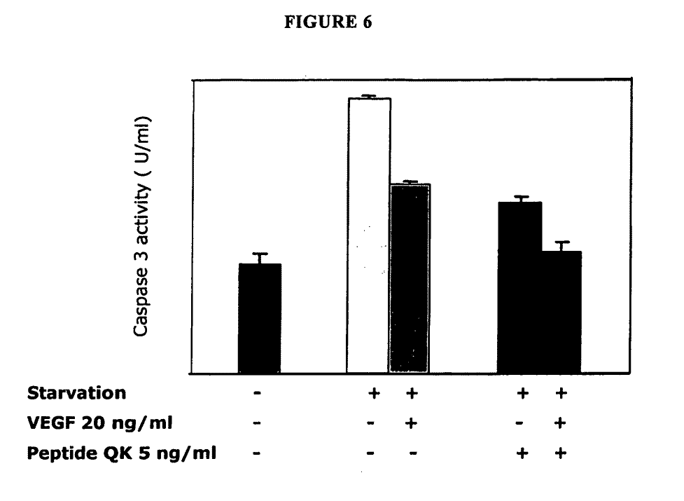

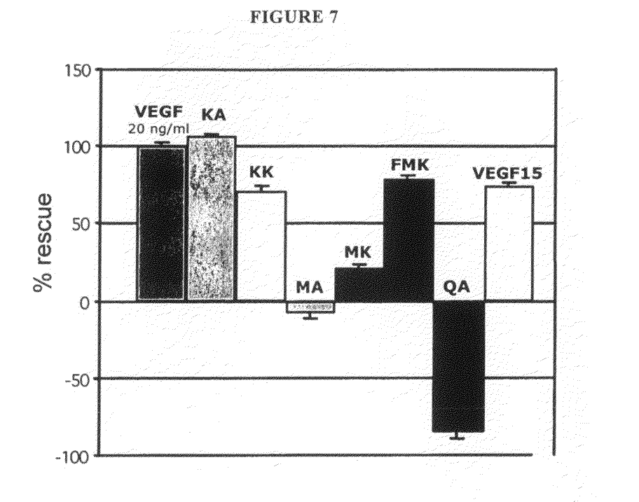

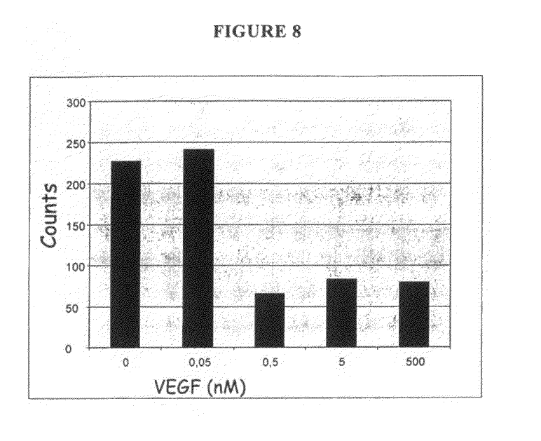

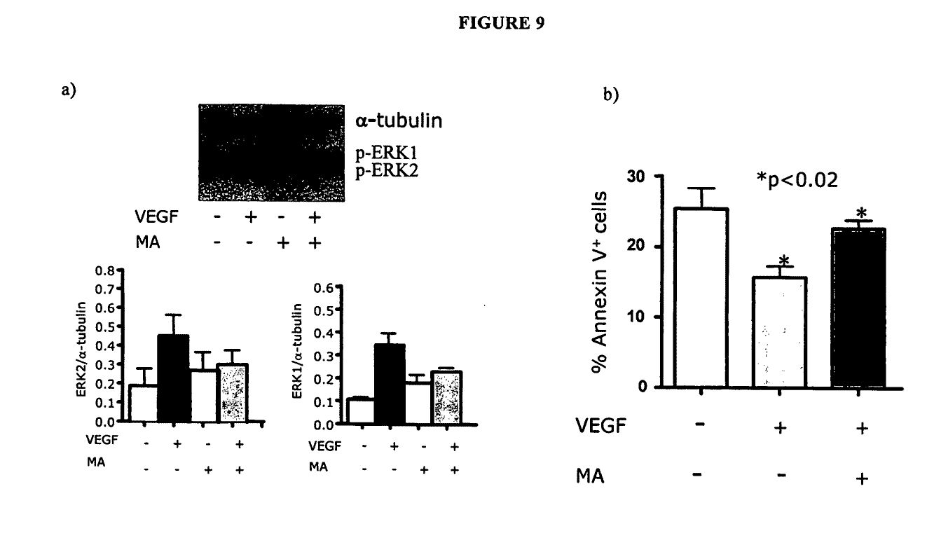

VEGF receptors binding assay. To verify the biological behavior of QK peptide, we tested its ability to compete for the binding sites of VEGF on cell membranes (FIG. 1a). We competed membranes, obtained from isolated BAEC, with iodinated VEGF and then with increasing amount of QK. Competition curves showed a displacement of iodinated VEGF by QK with an estimated apparent dissociation constant of 10^{-9.5} M, thus suggesting the interaction with receptors localized on particulate cellular fraction. To show that indeed VEGF receptors are involved in the binding to QK and to evaluate the ability of our compound to initiate early events of signal transduction, we immunoprecipitated total KDR and Flt-1 from BAEC whole extracts and visualized tyrosin phosphorylation by western blot. As expected 15 minutes of exposure to VEGF₁₆₅, used as control, caused the reduction in the levels of phospho-KDR at the membrane while increases Flt-1 phosphorylation (FIG. 1b,c) (6). QK exerted similar effects on these receptors, since it reduced phospho-KDR below the levels in unstimulated cells awhile increased the levels of phosphorylation of Flt-1. Together with ligand binding data, these results suggest that QK recapitulated the effects of VEGF₁₆₅ on VEGF receptors.

Activation of the proliferative intracellular pathways. We then explored whether QK is able to start the pathways of endothelial cell activation. It is well established that angiogenesis modulate by VEGF is largely ERK1/2 dependent, leading to DNA synthesis and cell proliferation (7). Accordingly, we assessed the effects of QK on this kinase. Indeed, QK leaded to ERK1/2 activation in a dose dependent fashion. This response was additive to VEGF, indicating that low doses of QK facilitate VEGF signaling (FIG. 2a). Instead, the peptide reproducing the natural helix (VEGF15) had no effect on ERK activation, proving that it is unable to start intracellular signaling (FIG. 2b). To verify whether ERK1/2 activation to QK results in cell proliferation, we studied cell proliferation indicators such as cell number, DNA synthesis and cyclin activation. QK increased DNA synthesis at any dosages and the effect was enhanced in presence of VEGF (FIG. 3a). Cell proliferation studies likewise indicated that QK produces cell proliferation per se and enhances VEGF response (FIG. 3b). Finally, QK and VEGF₁₆₅, but not VEGF15, enhanced phosphorylation of the cyclin RB, thus indicating cell cycle progression from G0 to G1 (FIG. 3c).

In vitro angiogenesis assay. To investigate whether QK recapitulates the overall angiogenic properties of VEGF, we studied the ability of the peptide to induce EC network formation on a matrigel substrate (FIG. 4). Tubule formation was evaluated by positive staining for CD31/PECAM-1, an intercellular adhesion molecule involved in leucocytes diapedesis. We determined the number of cell junctions corrected by the total tubules length. As positive control we used VEGF which caused an increase in the number of connections that each endothelial cell extend to the neighborhood cell (from 0.1±0.1 to 2.14±0.17). QK induced the formation of new connections in a dose dependent manner and enhanced the response to VEGF₁₆₅ (FIG. 4e).

In Vivo Angiogenesis Assay

We evaluated the proangiogenic effects of QK in vivo using a subcutaneous injection of Matrigel Matrix High (BD Technologies) containing or the control peptide (VEGF 15 10-7 M), in anesthetized twelve-week-old WKY rats. After one week the plugs were removed and analyzed in gross morphology and capillaries infiltration by CD31/vWF immunostaining. Plugs with QK at macroscopic inspection contained blood microscopic evaluation evidenced a greater peripheral capillaries infiltration in VEGF and QK plugs than in VEGF 15 plugs (data not showed). In another group of WKY we performed the ischemic hindlimb model associated with a chronic intrafemoral artery infusion of QK (10⁻⁷ M), VEGF (10⁻⁷ M) and VEGF 15 (10⁻⁷ M). After 14 days animals were anesthetized and hindlimbs blood flow (BF) assessed by digital angiographies counting the TIMI Frame score (TFC) needs to the contrast to arrive at the artery dorsal paw (FIG. 5a). BF was also evaluated by dyed beads dilution through injection in abdominal aorta of yellow beads (3*105) (FIG. 5b). By histology on the ischemic and non ischemic anterior tibial muscle, we evaluated capillary density. Data are presented in table and show the in vivo proangiogiogenic properties of the QK that are similar to VEGF, suggesting that also in vivo this peptide resemble the full protein.

| | | VEGF15 | VEGF165 | QK |
|---|---|---|---|---|
| ANGIO- | Number of TFC | 38 ± 2 | 18 ± 2 | 16 ± 2 |
| GRAPHY | | | | |
| BEADS | Ischemic to | 0.59 ± 0.15 | 0.97 ± 0.12 | 0.98 ± 0.12 |
| | non ischemic | | | |

Rescue from apoptosis of HUVE cells. To investigate whether the designed peptide was able to mimic the VEGF anti-apoptotic activity, we analyzed the activation of caspase 3 in human primary endothelial (HUVEC) cells deprived of FBS. The addition of VEGF partially rescued, as expected (Yilmaz A, et al., Biochem Biophys Res Commun. 2003, 306: 730), HUVEC cells from apoptosis. QK showed a biological effect similar to VEGF and enhanced the response of HUVEC to VEGF (FIG. 6).

Modulating angiogenesis in the adult life is a very attractive goal because it is involved in relevant pathological conditions. Therapeutic angiogenesis is sought as the ultimate intervention to solve chronic ischemia in those conditions that cannot be treated alternatively. Its converse, the anti-angiogenic treatment, is a promising therapy in oncology. Since the angiogenic response strictly depends on VEGF activity, this protein is considered a very attractive pharmacological target and in the last year it has been object of intense investigations.

The X-ray structure of the complex VEGF/Flt-1_{D2} shows that the binding interface is mainly localized in three regions (1). One of them is the α-helix spanning the amino acid sequence 17-25. We focused our attention on this region because it comprises some of the key residues involved in receptors recognition and because new molecules interacting with the receptors reproducing this region have not been developed so far. Moreover, the design of helical peptide represents a tractable target for peptide engineering since the folding and stability rules of helical peptides have been elucidated in the last years (5). It is well known that peptide fragments spanning the helices, turns and β-hairpins of natural proteins show little propensity, with very few exceptions, to reproduce their natural secondary structure under physiological conditions (5). Moreover, the stabilization of suitable conformational properties in aqueous solutions is a condition to gain the binding of designed peptides to their targets.

We reasoned that introducing appropriate tools in the natural sequence, such to stabilize the helical conformation, the key residues will be displayed in the three dimensional arrangements suitable for the receptor binding. We adopted a structure-based approach to design a linear peptide, QK, which should interact with the VEGF receptors. All the data collected about the structural preferences of the QK peptide in aqueous solution strongly indicated that it mainly folds in helical conformation. In particular, the first indication derives from the CD spectrum, which is well confirmed by the Hα chemical shift analysis and the NMR structure determination. These two latter analyses defined the QK helical region as that included between residue 4 and 12 which corresponds to the VEGF helical region and represents an important prerequisite for the QK biological activity. The stabilization of QK helical conformation is not a trivial result, as VEGF15 assumes in solution a random coil conformation, and because, typically, short peptides, composed of natural amino acids, are rarely helical in solution mainly due to inherent thermodynamic instability. The basis of the QK helical fold seems to reside on the presence of amino acids with intrinsic helix preference and on the amphipathic nature of the helix, which allows a number of medium range ionic, polar and hydrophobic interactions on opposite faces of the peptide. Moreover, QK peptide which is composed by only fifteen natural amino acids and whose structure in pure water has been derived with a good backbone resolution, could represent a model for further folding studies.

Most of the biological function of VEGF are mediated by its receptors KDR and Flt-1 (8-10). VEGF interaction with KDR or Flt-1 induces receptor dimerization and subsequent activation. Therefore, VEGF dimers are considered the only active form. In this paper we report evidences that the peptide QK binds to VEGF receptors. Binding studies showed that QK competes with VEGF for a binding site on endothelial cell membranes. These cells express both KDR and Flt-1 receptors, two tyrosine receptors that undergo autophosphorylative events upon binding to their agonist. To evaluate if QK has any preference towards one of the two receptors we immunoprecipitated the receptors and evaluated the tyrosin phosphorylation by western blot. Data reported in FIG. 1 showed that QK binds and activates both receptors similarly to VEGF. KDR phosphorylation decreases after ligand binding because KDR is internalized and digested, while Flt-1 remains exposed on the membrane (6). The agonist-like behavior of QK is confirmed by cell proliferation experiments and by the downstream activation of VEGF-dependent intracellular pathway (ERK1/2). It has been reported that VEGF stimulates DNA synthesis and proliferation in a variety of EC types (11-14). VEGF strongly induces the activity of ERK1/2 and the activation of this pathway presumably plays a central role in the stimulation of EC proliferation (15, 16). Our data showed that QK leaded to ERK1/2 activation and cell proliferation in a dose dependent fashion and in both experiments QK enhanced the VEGF activity. Moreover, we checked, as a marker of cell proliferation, the phosphorylation of RB, the cyclin that regulates proliferation by controlling progression through the restriction point within the G1 phase of the cell cycle. QK and VEGF₁₆₅, but not VEGF15, enhanced RB phosphorylation, thus indicating cell cycle progression from G0 to G1 (FIG. 3c).

We tested the biological properties of our peptide in a functional assay performing an in vitro angiogenesis assay using a matrigel substrate. VEGF is a potent angiogenic factor in vivo, which induces cell proliferation and migration through extracellular matrix to form threadlike tubule structures that join up to create a network of tubules (17, 18). QK, as shown in FIG. 4, induced the formation of new connections in a dose dependent manner and enhanced the VEGF response. This experiment confirmed that our peptide QK recapitulates many of the features in signal transduction that are reported for VEGF.

Finally, the pro-angiogenic properties of QK were demonstrated also in vivo using two different models. In both experiments the peptide QK showed an activity similar to VEGF whereas the control peptide, VEGF15, did not present any biological activity. The peptide QK showed also in vivo the ability to enhance the biological effects of VEGF. These data open new fields of investigation both on the mechanisms of activation of VEGF receptor and clinical implications.

We also showed that QK is able to rescue EC from apoptosis mimic the survival function of VEGF.

Overall, our results demonstrate that QK binds to VEGF receptors in vitro and show that it is a potential agonist for angiogenesis. A stable helical structure of the core region of the QK region appears to be a key requisite for its ability to bind the VEGF receptor. In fact, the natural fragment, VEGF15, which as expected is unstructured in water, did not show any appreciable biological activity alone or in combination with VEGF.

Therapeutic angiogenesis in cardiovascular conditions such as chronic ischemia or heart failure is sought as a promise of modern biotechnology. Indeed, the hypothesis that VEGF administration may result in therapeutically significant angiogenesis in humans has been already tested by Isner et al. (19) in a gene therapy trial in patient with severe limb ischemia. Major limitations to the use of growth factors such as VEGF are associated to their ability to promote uncontrolled neo-angiogenesis and lymphatic edema. Recent findings, furthermore, propose VEGF as a factor promoting asthma (20), a side effect that could preclude the use of this molecule in a large share of the ischemic population. Our data, are suggestive that either QK or improved analogues might fulfill the request for a safer pro-angiogenic drug.

### Example 2

Biological assays on Human Umbilical Vein Endothelial Cells (HUVEC) suggested that the peptide with the SEQ ID No. 3 and 5 (namely “MA” and “MK” respectively) are VEGF antagonists. Both are able to impair VEGF rescue of HUVEC from apoptosis. MA binds to the VEGF receptors and inhibits VEGF activation of ERK1/2 kinases.

Peptide Synthesis. Peptides were synthesized on solid phase using Rink Amide MBHA resin (Novabiochem) with standard Fmoc (N-(9-Fluorenyl)methoxycarbonyl) chemistry. The N-terminal lysine was protected with the methyltrytil group to allow selective deprotection and peptide labeling. Cleavage from the resin were achieved by treatment with trifluoracetic acid, triisopropyl silane, water, (95; 2.5; 2.5) at room temperature for 3 hours. Purity and identity of the peptides were assessed by HPLC and MALDI-ToF mass spectrometry.

Cells—Normal Human Umbilical Vein Endothelial Cells (HUVEC) were obtained from Promocell (Heidelberg, Germany) and cultured in EGM-2 SingleQuots (Cambrex, Carlsband, Calif.).

Analysis of caspase 3 activity—Cells (2×104) were lysed in a buffer containing Hepes 50 mM, DTT 1 mM, EDTA 0.1 mM, NP-40 0.1%, CHAPS 0.1% and protein quantitation determined. Protein aliquots (20 μg) were incubated with 20 μM Ac-DEVD-AMC (Pharmingen, San Diego, Calif.) in a buffer containing Hepes 50 mM, DTT 1 mM, EDTA 0.1 mM, NP-40 0.1%, CHAPS 0.1%, at 37° C. for 3 h. Caspase 3 activity was determined in the cytosolic extracts by analysing the release of 7-amino-4-methylcoumarin (AMC) from N-acetyl-DEVD-AMC (Thornberry N A, et al. Nature 1992; 356: 768-74); the release of AMC was monitored in a spectrofluorometer with an excitation wavelength of 380 nm and emission wavelength of 440 nm.

Human recombinant VEGF and other reagents—Human recombinant VEGF was obtained from R&D (Minneapolis, Minn.) Anti-phospho-ERK polyclonal antibody was obtained from Cell Signaling (Danvers, MA). Phycoerythrin (PE)-conjugated anti-β1-integrin monoclonal antibody (mAb) was obtained from Santa Cruz Biotechnology (Santa Cruz, Calif.). Fluorescein isothiocyanate (FITC)— conjugated annexin V was obtained from Bender MedSystems GmbH (Vienna, Austria). Anti-human α-tubulin mAb was obtained from Sigma (St. Louis, Mo.).

Fluorescence—Cells (3×105) were incubated with saturating amounts of FITC-conjugated and the other indicated reagents, 5 min at 4° C. in the dark. After washing with PBS, the cells were resuspended in PBS and analyzed with a FACScan (Becton Dickinson) flow cytometer.

Western blotting. Cell total protein lysates were prepared in sample buffer (2% sodium-dodecyl-sulphate, 10% glycerol, 2% mercaptoethanol and 60 mM Tris-HCl pH 6.8 in demineralized water) on ice. Lysates (25 μg) were run on 12% SDS-PAGE gels and electrophoretically transferred to nitrocellulose. Nitrocellulose blots were blocked with 5% BSA in Tris Buffer Saline Tween-20 (TBST) buffer [20 mM Tris-HCl (pH 7.4), 500 mM NaCl, and 0.01% Tween 20] and incubated with primary antibody in TBST-5% BSA overnight at 4° C. Immunoreactivity was detected by sequential incubation with horseradish peroxidase-conjugated secondary antibody and enhanced chemiluminescence reagents following standard protocols (Amersham Bioscience, UK).

Statistical analysis. Statistical analysis was performed using GraphPad Prism version 4.00 for Windows, GraphPad Software, San Diego, Calif., www.graphpad.com.

Results

Inhibition of VEGF activity by designed peptides. To investigate whether the designed peptides were able to compete with VEGF anti-apoptotic activity, we analyzed the activation of caspase 3 in human primary endothelial (HUVEC) cells deprived of FBS. While >20 U/ml of activated caspase 3 was evidenced in cell lysates from FBS-deprived cells, cells from cultures with VEGF appeared to contain <9 U/ml of the enzyme activity. Therefore the addition of VEGF partially rescued, as expected (Yilmaz A, et al., Biochem Biophys Res Commun. 2003, 306: 730-6), HUVEC cells from apoptosis. The effect of VEGF was abolished when MA peptide was added to the cultures; MK, partially inhibited the effect of the growth factor, while the other tested peptides were not able to modify VEGF activity (FIG. 7).

Binding of MA peptide to HUVEC cells. To verify that MA peptide bound to HUVEC cells, we incubated the cells with MA or a scrambled peptide conjugated with fluorescein and analyze the binding by FACS. MA peptide specifically bound to the cells in a dose-dependent manner (data not showed) and human recombinant VEGF, added to the cells, appeared to compete with MA peptide (FIG. 8).

Inhibition of VEGF-induced activation of ERK kinase by MA peptide. VEGF binding to HUVEC cells was shown to induce the activation of ERK kinase, resulting in inhibition of cell apoptosis (Salameh A, et al., Blood. 2005, 106:3423-31). We therefore investigated whether MA peptide binding to the cells could block ERK activation by VEGF. As shown in FIG. 9a, while cells stimulated with VEGF for 30 min displayed appreciable levels of the phosphorylated (activated) ERK1/2 kinase, these levels were highly reduced in cells incubated with VEGF in the presence of MA peptide.

Effect of MA peptide on the appearance of annexin V+ cells in cultures with VEGF. Apoptotic cells externalize phosphatidylserine, that is bound by annexin V (Steensma D P, et al., Methods Mol. Med. 2003; 85: 323-32). HUVEC cells deprived of FBS for 24 h displayed the 25% of annexin V+ cells; such percentage was >40% reduced in cultures with VEGF. In cells cultured with VEGF and MA peptide, we found >22% of annexin V+ cells (FIG. 9b), indicating that the peptide significantly (p<0.02) inhibited the anti-apoptotic affect of the growth factor.

### REFERENCES

1. Wiesmann, C., Fuh, G., Christinger, H. W., Eigenbrot, C., Wells, J. A. & de Vos, A. M. (1997) Cell 91, 695-704.
2. Muller, Y. A., L₁, B., Christinger, H. W., Wells, J. A., Cunningham, B. C. & de Vos, A.M. (1997) Proc Natl Acad Sci USA 94, 7192-7.
3. Keyt, B. A., Nguyen, H. V., Berleau, L. T., Duarte, C. M., Park, J., Chen, H. & Ferrara, N. (1996) J Biol Chem 271, 5638-46.
4. Aurora, R. & Rose, G. D. (1998) Protein Sci 7, 21-38.
5. DeGrado, W. F., Summa, C. M., Pavone, V., Nastri, F. & Lombardi, A. (1999) Annu Rev Biochem 68, 779-819.
6. Duval, M., Bedard-Goulet, S., Delisle, C. & Gratton, J. P. (2003) J Biol Chem 278, 20091-7.
7. Berra, E., Milanini, J., Richard, D. E., Le Gall, M., Vinals, F., Gothie, E., Roux, D., Pages, G. & Pouyssegur, J. (2000) in Biochem Pharmacol, Vol. 60,pp. 1171-8.
8. Ferrara, N. & Davis-Smyth, T. (1997) Endocr Rev 18, 4-25.
9. Shibuya, M. (2001) Cell Struct Funct 26, 25-35.
10. Zachary, I. & Gliki, G. (2001) Cardiovasc Res 49, 568-81.
11. Thakker, G. D., Hajjar, D. P., Muller, W. A. & Rosengart, T. K. (1999) J Biol Chem 274, 10002-7.
12. Pedram, A., Razandi, M. & Levin, E. R. (1998) J Biol Chem 273, 26722-8.
13. Xia, P., Aiello, L. P., Ishii, H., Jiang, Z. Y., Park, D. J., Robinson, G. S., Takagi, H., Newsome, W. P., Jirousek, M. R. & King, G. L. (1996) J Clin Invest 98, 2018-26.
14. Doanes, A. M., Hegland, D. D., Sethi, R., Kovesdi, I., Bruder, J. T. & Finkel, T. (1999) Biochem Biophys Res Commun 255, 545-8.
15. Wheeler-Jones, C., Abu-Ghazaleh, R., Cospedal, R., Houliston, R. A., Martin, J. & Zachary, I. (1997) FEBS Lett 420, 28-32.
16. Abedi, H. & Zachary, I. (1997) J Biol Chem 272, 15442-51.
17. Conway, E. M., Collen, D. & Carmeliet, P. (2001) Cardiovasc Res 49, 507-21.
18. Ferrara, N. (2001) Am J Physiol Cell Physiol 280, C1358-66.
19. Isner, J. M. & Asahara, T. (1999) J Clin Invest 103, 1231-6.
20. Lee, C. G., Link, H., Baluk, P., Horner, R. J., Chapoval, S., Bhandari, V., Kang, M. J., Cohn, L., Kim, Y. K., McDonald, D. M. & Elias, J. A. (2004) in Nat Med, Vol. 10, pp. 1095-103.

1-16. (canceled)

17. A method for regenerating blood vessels, comprising administering to a subject in need thereof, a compound mimetic of the VEGF helix spanning residues Phe17 through Tyr25, said compound being able to recognize VEGF receptor and to modulate both endothelial cell proliferation and angiogenesis.

18. The method according to claim 17, wherein the subject has an angiogenesis-dependent diseases.

19. The method according to claim 18, wherein said disease includes chronic ischemia, psoriasis, cancer, proliferative retinopathy and rheumatoid arthritis.

20. The method according to claim 19, wherein the subject has a tumor which is expressed on their surface VEGF receptors.

21. The method according to claim 20 wherein the subject has a lung tumor, thyroid tumor, breast cancer, gastrointestinal tumor, kidney tumor, ovarian tumor, uterine cervix tumor, carcinoma, angiosarcoma, germ cell tumor, or intracranial tumors.

22. The method according to claim 18, wherein the subject has an eye disease.

23. The method according to claim 22, wherein the eye disease is age related macular degeneration, diabetic retinopathy, vitreous hemorrhage, retinal detachment, or neovascular glaucoma.

24. The method according to claim 18 wherein the subject has a female-reproductive tract diseases.

25. The method according to claim 24 wherein the disease is ovarian hyperstimulation syndrome or endometriosis.

26. The method according to claim 18 for the treatment of brain edema, ischemic cardiovascular diseases, neuron disorders, bone diseases, bone disorders, gastric ulcer, diabetic foot ulcers, diabetic neuropathy, or wound healing.

27. A method for treating the over expression of VEGF receptors, comprising administering to a subject in need thereof a compound mimetic of the VEGF helix spanning residues Phe17 through Tyr25, and wherein said compound recognizes VEGF receptors and modulates both endothelial cell proliferation and angiogenesis.

28. The method according to claim 27, wherein the diagnostic composition is suitable for the imaging of angiogenic vasculature.

29. A method for studying cellular pathways dependent on VEGF receptor activation, comprising testing and studying the effects of compound mimetic of the VEGF helix spanning residues Phe17 through Tyr 25 on said pathway and wherein said, compound recognizes VEGF receptors and to modulates both endothelial cell proliferation and angiogenesis.

30. The method according to claim 17, wherein said compound is a peptide selected from one of SEQ ID No. 1 to SEQ ID No.8:

31. A method for treating a subject affected by angiogenesis-dependent pathologies comprising administering to said subject an effective amount of a compound mimetic of the VEGF helix spanning residues Phe17 through Tyr25, and wherein said compound recognizes VEGF receptors and modulates both endothelial cell proliferation and angiogenesis.

32. The method according to claim 31, wherein said compound is a peptide selected from one of SEQ ID no. 1 through SEQ ID No. 8.
